(19) United States
(12) Patent Application Publication
(10) Pub. No.: US 2013/0302872 A1
(43) Pub. Date: Nov. 14, 2013

(54) PRODUCTION OF CELL TISSUE HAVING THREE-DIMENSIONAL STRUCTURE USING ELECTROSTATIC INK JET PHENOMENON

(71) Applicant: RIKEN, Saitama (JP)

(72) Inventors: Shinjiro UMEZU, Saitama (JP); Takashi KITAJIMA, Saitama (JP); Kazutoshi KATAHIRA, Saitama (JP); Hitoshi OHMORI, Saitama (JP); Yoshihiro ITO, Saitama (JP)

(73) Assignee: RIKEN, Saitama (JP)

(21) Appl. No.: 13/947,142

(22) Filed: Jul. 22, 2013

Related U.S. Application Data
(62) Division of application No. 13/054,475, filed on Jan. 14, 2011.

(30) Foreign Application Priority Data
Jul. 17, 2008 (JP) 2008-186068

(51) Int. Cl. C12N 13/00

(52) U.S. Cl. CPC C12N 13/00
USPC 435/173.1

(57) ABSTRACT

A method and an apparatus for producing a cell tissue of blood vessels or the like with the use of an electrostatic ink jet phenomenon are provided. A pattern generator for cell tissue patterning on a substrate includes at least one vessel for holding a solution containing materials used for cell tissue formation, a substrate that is an object of patterning, and a pattern generator including a voltage applying unit for applying a voltage to the vessel. The voltage is applied to the vessel by the voltage applying unit to generate an electric field between the vessel and the substrate, causing the solution to be ejected from the vessel as a result of an electrostatic ink jet phenomenon and allowing the ejected solution to adhere to the substrate.

### CROSS-REFERENCE TO RELATED APPLICATION

The present application is a divisional of application Ser. No. 13/054,475, filed Jan. 14, 2011, the contents of which are incorporated herein by reference.

### TECHNICAL FIELD

The present invention relates to a method for producing a cell tissue having a three-dimensional structure using an electrostatic ink jet phenomenon and an apparatus therefor.

### BACKGROUND OF THE INVENTION

In biotechnology studies on regenerative medicine, artificial organs, and the like, it has been strongly desired to use a cell tissue having a three-dimensional structure that is similar to that of a real tissue for experimentation. If a tissue having a three-dimensional structure is artificially formed, it is required that such artificial tissue actually function as a tissue. However, the weight of a tissue having a three-dimensional structure exceeds the cell-to-cell binding force. Therefore, even in the case of a cell tissue comprising cells aligned in a three-dimensional form, the shape thereof cannot be maintained, and thus it is deformed so as to have a two-dimensional form. For such reason, it has not been easy to artificially produce a tissue having a three-dimensional structure.

Hitherto, methods have existed for constructing a biological tissue having a three-dimensional structure in an ex vivo environment. For example, a method comprising introducing a scaffold having a three-dimensional structure that serves as a cell foothold into a liquid containing cells so as to allow the cells to adhere to the surface of the scaffold has been suggested. An existing example of such method is a method comprising allowing cells capable of differentiating to form a bone to proliferate on a scaffold formed with calcium phosphate, a poly(lactic-co-glycolic acid) (PLGA) copolymer, or the like in the presence of a cell growth factor so as to use the obtained bone as an artificial bone (see Non-Patent Document 1). However, according to such technique, it is merely possible to allow cells of a single type to adhere to a scaffold that has been previously formed into a given shape and to proliferate thereon. Therefore, it has been impossible to freely carry out structural patterning of a plurality of types of cells.

In addition, there is a technique for producing an artificial tissue by patterning alginic acid capsules encapsulating cells in a two-dimensional or three-dimensional form via a conventional ink jet technique using a piezoelectric system or a thermal system (see Non-Patent Document 2). According to such ink jet technique using a piezoelectric system or a thermal system, ejection of a highly viscous solution cannot be controlled. Therefore, cells are encapsulated in alginic acid capsules so that they can be used. However, such technique is problematic in that cell-to-cell adhesion and interaction are prohibited by alginic acid capsules. Accordingly, even though cells are aligned in a two-dimensional or three-dimensional form in the above case, the obtained tissue cannot function as a tissue. According to the above technique based on an ink jet technique, patterning of cells can be freely carried out to some extent. However, it cannot be said that such technique is used as a practical patterning method.

The present inventors studied electrostatic ink jet phenomena via which liquid ejection is controlled by controlling electrostatic force and thus developed an electrostatic ink jet technique, which is a novel ink jet technique that differs from the above technique using a piezoelectric system or a thermal system (see Non-Patent Document 3). This technique requires application of a high voltage to liquid. Therefore, it was thought living cells used in such case would be killed.

Non-Patent Document 1: YANG, X. et al., Journal of Bone and Mineral Research, 18:47-57, January 2003

Non-Patent Document 2: Henmi C. et al., AATEX, 14, Special Issue, 689-692, 2008.

Non-Patent Document 3: Kawamoto H. et al., JOURNAL OF IMAGING SCIENCE AND TECHNOLOGY, Volume 49, Number 1, January/February 2005, pp. 19-27

### SUMMARY OF THE INVENTION

It is an object of the present invention to provide a method and an apparatus for producing a cell tissue of blood vessels or the like with the use of an electrostatic ink jet phenomenon. Cell tissue obtained via accurate two-dimensional or three-dimensional patterning is necessary in the field of biotechnology related to regenerative medicine/artificial organs. The present invention enables such cell tissue patterning.

In order to solve the above problems posed by conventional techniques, a technique for patterning cells and scaffolds that serves as cell footholds is required. For such scaffolds, an extracellular matrix material is desirably used. However, it is necessary for such material to be highly viscous so as to obtain the strength sufficient for it to be used as footholds. However, even if an ink jet printer with a piezoelectric system or a thermal system is modified such that ejection power can be slightly improved, it is still difficult to stably eject highly viscous scaffolds. Therefore, it has been impossible to produce a tissue having a three-dimensional structure that can function as a real tissue.

The present inventors conducted intensive studies in order to produce a cell tissue having a three-dimensional structure for blood vessels or the like with the use of the previously developed electrostatic ink jet technique using an electrostatic ink jet phenomenon. According to conventional ink jet techniques using a piezoelectric system or a thermal system, it has been impossible to control the amount of ink to be ejected or the ejection pattern when using a highly viscous liquid as ink. That is, a liquid containing cells is particularly highly viscous and therefore causes a capillary tube used for an ink jet printer to be clogged, making it impossible to stably eject the liquid.

The present inventors found that a highly viscous liquid containing scaffolds and cells can be stably ejected without clogging with the use of an electrostatic ink jet phenomenon, and that cells can be ejected without being killed even when a high voltage is applied to the cells. They succeeded in producing a cell tissue having a three-dimensional structure with the use of an electrostatic ink jet phenomenon. This has led to the completion of the present invention.

Specifically, the present invention is described below.
[1] A pattern generator for cell tissue patterning on a substrate, which comprises

at least one vessel for holding a solution containing materials used for cell tissue formation,

a substrate that is an object of patterning, and

a voltage applying unit for applying a voltage to the vessel, wherein a voltage is applied to the vessel by the voltage applying unit in order to generate an electric field between the vessel and the substrate, causing the solution to be ejected from the vessel as a result of an electrostatic ink jet phenomenon and allowing the ejected solution to adhere to the substrate.
[2] The pattern generator according to [1], wherein the substrate is grounded and a voltage is applied to the vessel by the voltage applying unit, causing the solution to be ejected from the vessel as a result of an electrostatic ink jet phenomenon and allowing the ejected solution to adhere to the substrate.
[3] The pattern generator according to [1], which further comprises a plate electrode that is positioned between the vessel and the substrate, grounded, and perforated, wherein a voltage is applied to the vessel by the voltage applying unit, causing the solution to be ejected from the vessel as a result of an electrostatic ink jet phenomenon and allowing the ejected solution to pass through a hole formed in the plate electrode and adhere to the substrate.
[4] The pattern generator according to any one of [1] to [3], wherein at least one vessel for holding a solution containing materials used for cell tissue formation is a vessel for holding a solution containing scaffolds that serves as cell footholds and a vessel for holding a solution containing cells.
[5] The pattern generator according to any one of [1] to [4], which further comprises a vessel for holding a solution containing a cell growth factor.
[6] A method for producing a cell tissue having a three-dimensional structure by patterning a cell tissue on a substrate using the pattern generator according to any one of [1] to [3], which comprises a step of ejecting scaffolds and cells onto a substrate so as to allow the scaffolds and the cells to adhere to the substrate for patterning, wherein a cell tissue having a three-dimensional structure, in which neighboring cells are adhering (directly or via an extracellular matrix material) to each other and the three-dimensional structure is maintained by the scaffolds, is produced.
[7] A method for producing a cell tissue having a three-dimensional structure by patterning a cell tissue on a substrate according to [6], which comprises the following steps of:

(i) ejecting scaffolds onto the substrate for patterning; and

(ii) carrying out patterning by ejecting cells in a manner so as to allow the cells to come into contact with the patterned scaffolds, wherein

a cell tissue having a three-dimensional structure, in which neighboring cells are in contact with each other and the three-dimensional structure is maintained by the scaffolds, is produced.
[8] The method for producing a cell tissue having a three-dimensional structure according to [6] or [7], wherein the three-dimensional structure is formed by repeating patterning of scaffolds and cells so that a plurality of layers of scaffolds and cells are accumulated.
[9] The method for producing a cell tissue having a three-dimensional structure according to any one of [6] to [8], which further comprises ejecting a cell growth factor in a manner so as to allow the cell growth factor to come into contact with cells.
[10] The method for producing a cell tissue having a three-dimensional structure according to any one of [6] to [9], wherein two or more types of cells are used.
[11] The method for producing a cell tissue having a three-dimensional structure according to any one of [6] to [10], wherein the voltage applied to the vessel for ejection of scaffolds and cells is 0.5 kv to 5 kv.
[12] The method for producing a cell tissue having a three-dimensional structure according to any one of [6] to [11], wherein the temperature of the vessel for holding a scaffold is maintained at 37° C. or higher and the temperature of the surface of the substrate is maintained at 37° C. or lower.
[13] An artificial cell tissue having a three-dimensional structure, which is produced by the method for producing a cell tissue having a three-dimensional structure according to any one of [6] to [12], in which cells remain alive, neighboring cells are adhering to each other, and a three-dimensional structure is maintained by scaffolds.

This description includes part or all of the contents as disclosed in the description and/or drawings of Japanese Patent Application No. 2008-186068, which is a priority document of the present application.

### BRIEF DESCRIPTION OF THE DRAWINGS

FIG. 1 schematically shows the configuration of a pattern generator 1 that allows patterning of cells or a biomaterial on a substrate using an electrostatic ink jet system.

FIG. 2 shows a graph indicating the relationship between a corona discharge current that flows during cell patterning and an applied voltage.

FIG. 3 schematically shows the configuration of an improved pattern generator 2.

FIGS. 4A to 4D each show conditions of cell lines and cells subjected to drawing by a pattern generator. FIGS. 4A and 4B each show cell lines. FIG. 4C shows cells subjected to drawing. FIG. 4D shows an enlarged view of cells subjected to drawing.

FIGS. 5A and 5B each show a design for a three-dimensional tissue containing cells and scaffolds produced by a pattern generator.

### EXPLANATION OF REFERENCE NUMERALS

1: Pattern generator
11: Scaffold syringe
12: Cell syringe
13: Capillary tube
14: Dish
15: Stage unit
16: Stage driver
17: High voltage amplifier
18: Oscillator
19: Control unit
2: Improved pattern generator
21: Plate electrode

### BEST MODE FOR CARRYING OUT THE INVENTION

The present invention is described in detail below.

### <Configuration of a Pattern Generator>

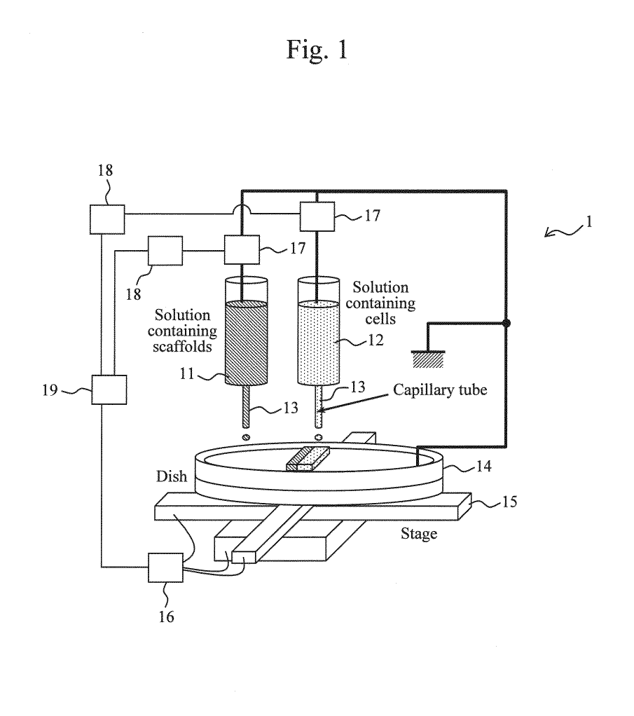

FIG. 1 schematically shows the configuration of a pattern generator 1 that allows patterning of cells or a biomaterial on a substrate using an electrostatic ink jet system. A pattern generator 1 is provided with the following: a scaffold syringe 11, which is a vessel for holding a solution containing scaffolds that serve as cell footholds (e.g., gelatin); a cell syringe 12, which is a vessel for holding a solution containing cells; a capillary tube 13 attached to each syringe; a dish (substrate) 14 for holding a substrate that is an object of patterning; a stage unit 15 for transferring a dish 14 from one position to another; a stage driver 16 for controlling the transfer of a stage unit 15 in accordance with an order from a control unit 19; a high voltage amplifier 17 and an oscillator 18 for applying a high voltage to a syringes 11 and 12; and a control unit (composed of, for example, a computer) 19 for controlling an applied voltage and the transfer of a stage. Here, a dish 14 is grounded and thus an electric field is formed between syringes 11 and 12 and a dish 14 as a result of application of high voltage.

A voltage waveform generated by a control unit 19 and an oscillator 18 is amplified by a high voltage amplifier 17 in a syringe 11 or 12 for patterning of scaffolds and cells. In addition, patterning is carried out at an applied voltage of 0.5 kv to 5 kv.

The intensity of an electric field formed between capillary tubes 13 and a dish 14 is controlled by controlling the intensity of the applied voltage. The amount of liquid ejected from each capillary tube (nozzle) 13 is adjusted by controlling the electric field intensity. Liquids (a scaffold solution and a cell solution) to be ejected are electrically charged with an applied voltage. Therefore, it is desirable that at least the ejection face of the tip of each nozzle be insulated. Accordingly, as described in this embodiment, a glass capillary tube is desirably used as such nozzle.

In addition, a stage driver 16 transmits transfer signals to a stage 15 under the control of a control unit 19 such that the position of a dish 14 is controlled. Specifically, scaffolds are first ejected so as to adhere to a dish 14 for patterning in a desired shape. Further, cells are allowed to adhere to the side or the top face of the scaffolds for patterning. After drawing of a single layer, the height of a culture solution filling the dish is increased by the height of a single layer. This operation is carried out repeatedly such that cell tissues are accumulated in a three-dimensional form.

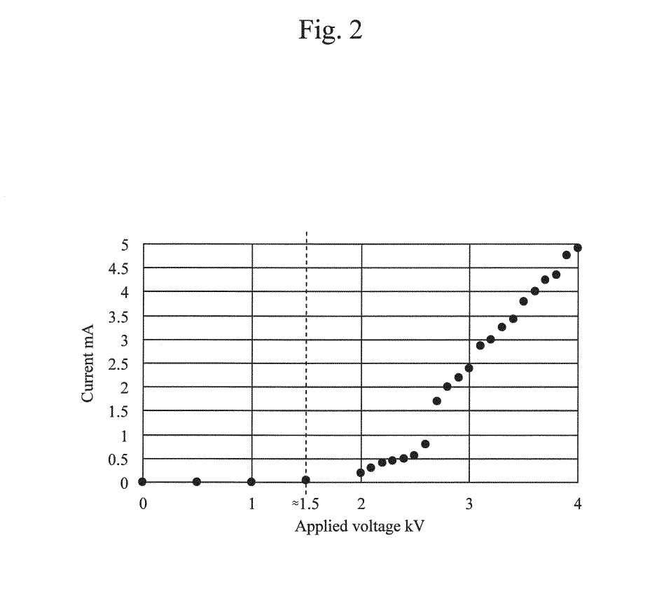

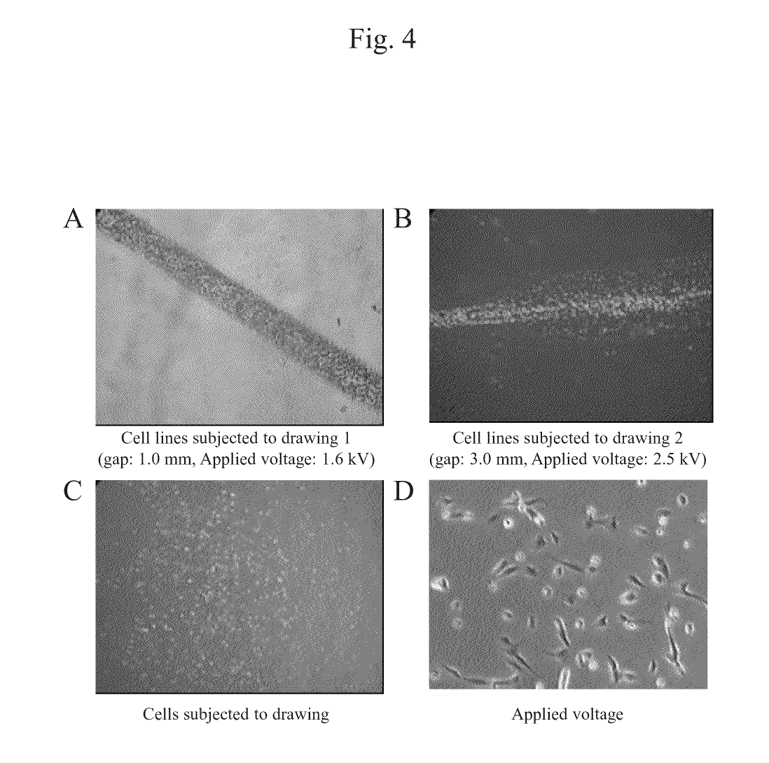

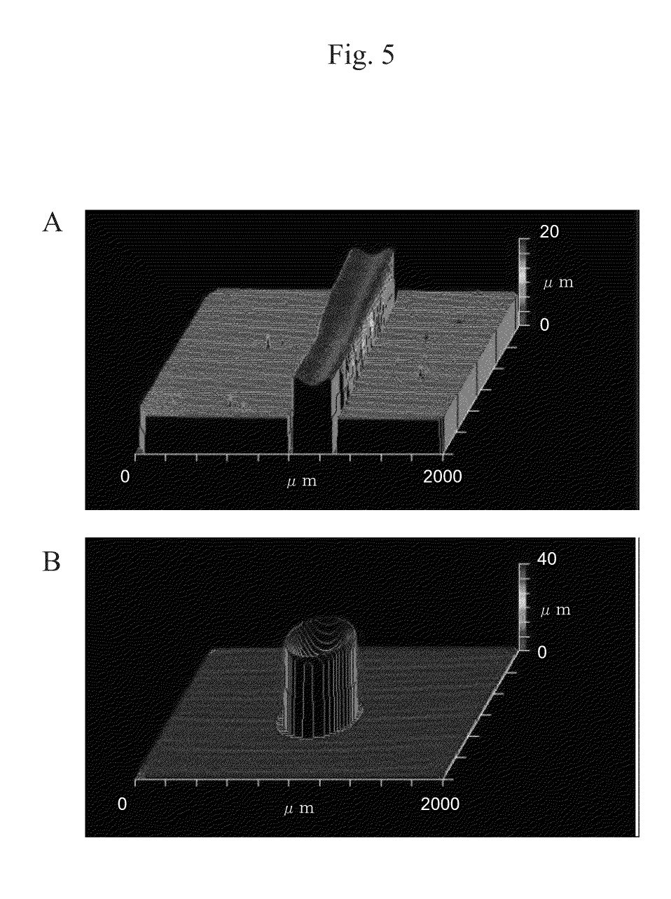

FIG. 2 shows a graph indicating the relationship between a corona discharge current that flows upon cell patterning and an applied voltage. It was experimentally confirmed that a Taylor cone is formed during ejection of a liquid at an applied voltage of approximately 1.5 kv or higher. Also, it was experimentally confirmed that the dot diameter of a liquid ejected becomes smaller as the applied voltage becomes closer to 1.5 kv. As described above, a Taylor cone is formed in a triangular pyramid shape on the tip of syringe 11 or 12 when a high voltage of 1.5 kv or higher is applied. The tip portion of the Taylor cone was exclusively removed and ejected in a droplet form. At such time, the gap (between the syringe and the dish) is set at 0.5 mm so as not to cause spark discharge. In such a case, if the gap exceeds 10 mm, floating droplets are swept away by the surrounding air flow, resulting in reduction of patterning accuracy. In the above case, although a high voltage is applied, cells remain alive without being killed after deposition. Also, contact between ejected cells has been confirmed (see FIGS. 4A to 4D). With the use of the above technique, a cell tissue can be three-dimensionally formed as shown in FIG. 5. In addition, as a capillary tube for ejecting cells, a capillary tube with an inner diameter of at least 30 μm and preferably 30 μm to 100 μm can be used. The inner diameter of a capillary tube for ejection of scaffolds and a cell growth factor is 10 μm to 100 μm. In view of cell size, the droplet diameter is at least one-tenth but less than half of the outer diameter of such tube. The apparatus of the present invention allows patterning in a more accurate manner than is possible with commercially available ink jet printers using a piezoelectric system or a thermal system. In addition, a liquid in a paste form having a viscosity 6000 times greater than that of a pigment ink used for a commercially available ink jet printer can be ejected, realizing three-dimensional design.

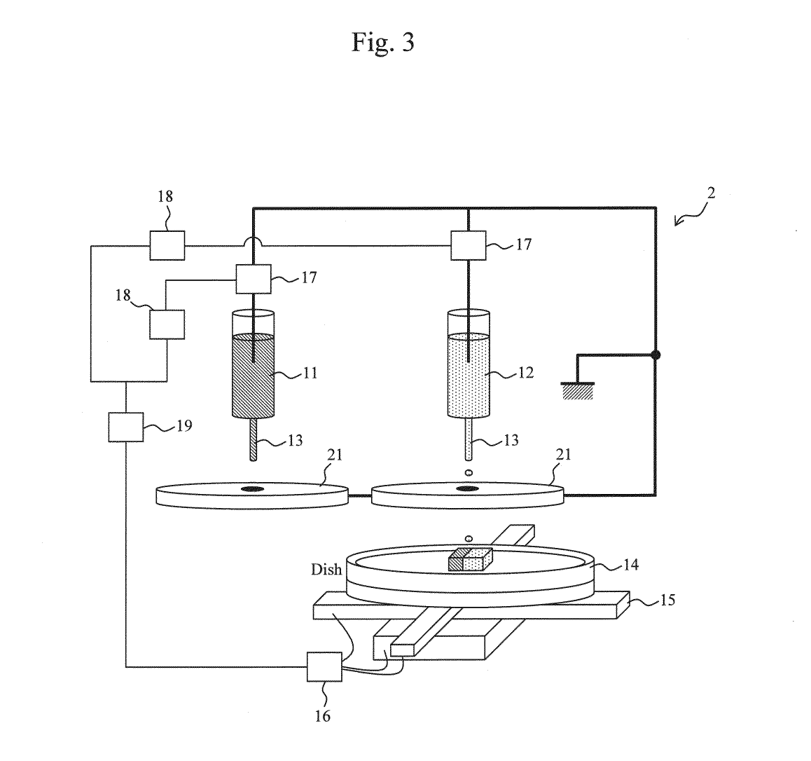

The basic configuration and the movement of the pattern generator of the present invention are described above. Next, an improved example of the pattern generator is described below. FIG. 3 schematically shows the configuration of an improved pattern generator 2.

In the case of the configuration shown in FIG. 1, an electric field is formed between capillary tubes 13 and a dish 14 by voltage application, resulting in concentration of an electric field at the edge portion of a formed pattern (electric field edge effects). This might, in some cases, prevent scaffolds and cells from being uniformly accumulated.

Such problem can be resolved according to the configuration of an improved example shown in FIG. 3. A pattern generator 2 is provided with a plate electrode 21 having a through hole for the passage of ejected liquid between a capillary tube 13 and a dish 14, in addition to the configuration shown in FIG. 1. Further, in a pattern generator 2, a voltage is applied between syringes 11 and 12 and a plate electrode 21 but not between the syringes and the dish. As a result of such configuration, an electric field is exclusively formed between a capillary tube 13 and a plate electrode 21. Accordingly, the influence of an electric field upon a dish 14 can be removed. Specifically, even in a case in which cells are accumulated to a great height in a three-dimensional form, the electric field formed around the capillary tube tip does not vary, and therefore the ejection pattern is unlikely to be changed. As a result, it becomes possible to uniformly accumulate scaffolds and cells on a substrate. Thus, effects of facilitating formation of a desired pattern are obtained. In addition, there is a probability that the threshold voltage value for Taylor cone formation slightly would differ from the voltage of approximately 1.5 kv shown in FIG. 1. However, it is certain that the value is close to approximately 1.5 kv. It is believed that the relationship between the corona discharge current and the applied voltage tends to become similar to that shown in FIG. 2.

### <Materials for Production of Three-Dimensional Tissue>

According to the method of the present invention, materials used for formation of cell tissue are ejected onto a substrate. Examples of a substrate include glass, resins, vessels made of metals or the like, flat plates, film, and membranes. Specifically, a culture vessel such as a culture dish, a glass slide, or the like can be used.

Examples of materials used for formation of cell tissue include scaffolds that serve as cell footholds, cells, and a cell growth factor. A plurality of such materials may be contained in a solution.

A material that constitutes an extracellular matrix in a biological tissue can be used for scaffolds. An extracellular matrix refers to a stable biological construct that is present outside a cell in biological tissue. It has cell adhesion activity and functions as a structural support when cells form a three-dimensional structure. In addition, an extracellular matrix has an action of controlling cell shaping, cell transfer, cell proliferation, intracellular metabolism, cell differentiation, and the like. Examples of a material that constitutes an extracellular matrix include collagen, elastin, proteoglycan, glycosaminoglycan, and glycoprotein. Examples of glycoprotein include fibronectin, laminin, and vitronectin. Therefore, for scaffolds, an aforementioned material and gelatin that is a denatured material formed from collagen can be used. In addition, cadherin, secretin, or a material belonging to the immunoglobulin superfamily such as NCAM or ICAM can be used for scaffolds, since it has an action of connecting cell membranes. Further, fibrinogen and fibrin can be used for scaffolds. Fibrinogen is formed into fibrin via a cleavage reaction in the presence of thrombin during bleeding so as to be polymerized. Upon polymerization, fibrin forms a fibrin clot together with other plasma proteins and blood cells for wound adhesion, exhibiting hemostatic effects. Fibrinogen and thrombin purified from blood are mixed, and the mixture is used as a hemostatic agent (fibrin glue). It is known that such materials can function as cell footholds because they have a function to cause tissue adhesion and exhibit a certain level of strength. In some cases, such material is called an “artificial matrix,” and it is produced for temporary use. A single type of material described above may be used for scaffolds. Alternatively, two or more types of materials described above may be mixed together and used. Such a material can be extracted from living organisms, produced using cells, or artificially synthesized.

The concentration of an aforementioned material ejected by a pattern generator using an electrostatic ink jet system is not limited, and it can be appropriately determined depending on the material. Such a material can be used at a concentration of, for example, 0.1 to 10 mg/ml. A scaffold may be dissolved in physiological saline, a medium, a buffer, or the like before use.

Cells used in the present invention are not limited and thus can be appropriately selected depending on the type of tissue to be produced. For example, differentiating cells or stem cells from the ectoderm, mesoderm, or endoderm can be used. More specifically, for example, vascular endothelial cells, epidermal cells, smooth muscle cells, osteocytes, chondrocytes, skeletal myoblasts, pancreatic parenchymal cells, pancreatic ductal cells, hepatocytes, blood cells, cardiomyocytes, skeletal muscle cells, osteoblasts, neurons, pigment cells, and adipocytes can be used. In addition, examples of stem cells that can be used include: tissue stem cells such as epidermal stem cells, follicle stem cells, common pancreatic stem cells, liver stem cells, nerve stem cells, retinal stem cells, hematopoietic stem cells, and mesenchymal stem cells; embryonic stem cells (ES cells); and iPS cells (induced pluripotent stem cells).

When a produced tissue is used for transplantation, it is preferable to use autologous cells from an individual (recipient) or cells from an individual whose major histocompatibility antigen type is identical or homologous to that of a recipient in order to prevent transplantation rejection.

Such cells are selected depending on the type of tissue to be produced. However, when a tissue comprising a plurality of types of cells is produced, a plurality of different cells can be used. That is to say, two or more types of cells can be used. For instance, when blood vessels are formed, vascular endothelial cells and smooth muscle cells can be used. Cells that are isolated from a biological tissue and cultured can be used. Alternatively, cells obtained via differentiation/proliferation of the above stem cells can be used. The cell density for ejection by a pattern generator using an electrostatic ink jet system is not limited. For instance, cells can be used at a density of 10⁴ to 10⁸ cells/ml. Cells can be suspended in a medium used for conventional cell culture such as an MEM medium, an α MEM medium, a DME medium, a BME medium, an IMEM medium, an RPMI medium, or an ES medium, or in physiological saline, buffer, or the like before use.

Further, a cell growth factor and cytokine can be used for cell tissue production. The term “cell growth factor” used herein refers to a material that promotes or controls cell proliferation. The term “cytokine” used herein refers to a physiologically active substance that is produced from cells and acts on cells from the same or different species. Examples of a cell growth factor include a platelet-derived growth factor (PDGF), an epidermal growth factor (EGF), a fibroblast growth factor (FGF), a hepatocyte growth factor (HGF), a vascular endothelial growth factor (VEGF), and insulin. In addition, examples of cytokine include: hematopoietic factors such as interleukins, chemokines, and colony-stimulating factors; tumor necrosis factors; and interferons. A cell growth factor can be used depending on the type of cells to be used. In addition, examples of other physiologically active substances that can be used include: vitamins such as ascorbic acid, biotin, calcium pantothenate, and vitamin D; proteins such as transferrin, and serum albumin; lipid; linoleic acid; cholesterol; pyruvic acid; retinoic acid; and antibiotics. In the present invention, the above materials, which are effective for cell proliferation, are collectively referred to as cell growth factors. In addition, in the present invention, the above scaffolds, cells, and cell growth factors may be referred to as biomaterials.

### <Production of Three-Dimensional Tissue>

According to the method of the present invention, a cell tissue having a three-dimensional structure is produced by ejecting scaffolds, cells, and cell growth factors described above onto a substrate in a specific pattern via a pattern generator using an electrostatic ink jet system. The term “cell tissue” used herein refers to a tissue composed of cells, as a main component. The tissue structure thereof is maintained by scaffolds that serves as cell footholds, and adhesion between cells is observed therein. The following forms of cell adhesion (or simply adhesion) exist: cell-cell adhesion and cell-matrix adhesion. In the present invention, cells do not necessarily adhere directly to each other immediately after having been ejected. However, it is important for an extracellular matrix or an artificial matrix to exist between cells. Such mediating material is materials (scaffolds) that serve as footholds. It maintains the structural intensity of formed tissue and supports cell survival. In addition, it does not prevent contact or subsequent adhesion between cells, or it has a function to actively promote such cell contact or adhesion. Cell-matrix adhesion is achieved by integrin. Meanwhile, cell-cell adhesion is the formation of an adhesion device between cells. The term “adhesion device” refers to a structure such as an adherence junction, a tight junction, a gap junction, a desmosome, or a hemidesmosome. Such device is formed by, for example, a cellular surface protein (an adhesion protein such as cadherin or a channel protein such as connexin) and an intracellular cell skeleton. Cells secrete a material that degrades a matrix so as to partially remove a mediating material. Thus, cells obtain surfaces that can come into contact with each other. This causes progression of adhesion, resulting in completion of a tissue structure. That is, according to the present invention, it is particularly desirable to print a material that serves as scaffolds and cells simultaneously or alternately.

A pattern generator using an electrostatic ink jet system has a single or a plurality of vessel(s) (syringe(s)) for accommodating liquid to be ejected. A plurality of syringes are allowed to separately hold a scaffold solution, a cell suspension, and a cell growth factor (cell proliferation factor) solution and then used. In addition, a single syringe may hold a plurality of materials such as cells and scaffolds, cells and a cell growth factor, or scaffolds and a cell growth factor. In addition, when a plurality of cells are used, a plurality of syringes that are allowed to hold cell suspensions may be used in a manner such that the syringes separately hold different types of cells.

A liquid in a syringe is allowed to pass through a capillary tube attached to the syringe so as to be ejected onto a substrate. For ejection of a solution in a syringe, a voltage is applied from a voltage applying unit of a pattern generator to a vessel. At such time, the applied voltage is 0.5 kv to 5 kv. When cells are ejected, a current of several microamperes flows among cells. However, it does not cause fatal damage to cells because the current flows outside the cells. The pattern generator of the present invention can produce a cell tissue having a three-dimensional structure in which cells remain alive without being killed even after the application of high voltage. The ejection pattern can be appropriately controlled depending on the tissue to be produced. For instance, scaffolds qre ejected onto a substrate in a given pattern so as to adhere to the substrate such that scaffolds with a given shape is formed on the substrate. Next, cells are ejected in a given pattern onto the side portion or the top face of the scaffold so as to come into contact with the scaffolds, and thus cells are allowed to adhere to the substrate. Here, adhesion to a substrate includes direct adhesion to a substrate and subsequent adhesion to a substrate via a scaffold or cells adhering to the substrate. For such adhesion, it is desirable for a single droplet ejected from a pattern generator using an electrostatic ink jet system to contain a single cell. The diameter of a droplet to be ejected is determined to be several to several tens of micrometers. In addition, if necessary, ejection is carried out in a manner that allows a cell growth factor to come into contact with cells. After ejection of scaffolds and cells for formation of a single layer onto a substrate, the second layer is formed in the same manner. At such time, it is preferable to increase the height of a substrate by the thickness of a single layer such that the distance between the position of the tip of a capillary tube for liquid ejection and the position of the site of the substrate onto which cells and the like are ejected is constantly maintained. In such case, the thickness of a single layer is determined based on the cell diameter and the scaffolds thickness. The thickness is approximately 1 to 50 μm. The above step is carried out repeatedly. Accordingly, a tissue having a three-dimensional structure in which a plurality of layers of cells and scaffolds are accumulated can be produced. Here, patterning may be carried out in a manner such that cells and scaffolds are alternately disposed (in the tissue thickness direction). Alternatively, patterning may be carried out in a manner such that scaffolds cover cells. The ratio of cell volume to scaffolds volume, which in turn indicates the amount (mass) of scaffolds per cell or the like, can be appropriately determined depending on the type of cell tissue to be produced.

The temperatures of syringes holding a cell suspension, scaffolds, and a cell growth factor are preferably maintained at 37° C. or higher. In particular, scaffolds becomes highly viscous when the temperature decreases. Therefore, it is desirable to maintain the temperature of the syringes at 37° C. or higher to cause scaffolds to become liquidified. In addition, the temperature of a syringe holding a cell suspension is preferably maintained at 40° C. or lower in order to prevent killing of cells. Further, the temperature of a substrate is desirably maintained at 37° C. or lower such that scaffolds ejected onto a substrate is likely to become solidified after deposition. In addition, the temperature of a substrate is desirably maintained at 40° C. or lower in order to prevent killing of cells.

A pattern for patterning can be controlled by a computer connected to a pattern generator.

For instance, when blood vessels are produced, cell patterning can be carried out in a manner that allows smooth muscle cells to cover vascular endothelial cells. In such case, vascular endothelial cells serve as cells that constitute the inner wall and smooth muscle cells serve as cells that constitute the outer wall.

According to the method of the present invention, a variety of artificial cell tissues, artificial body parts, and artificial organs can be produced. Examples thereof include skin, blood vessels, myocardia, muscles, corneas, kidneys, hearts, livers, umbilical cords, intestines, nerves, lungs, placenta, pancreases, brains, joints, bones, cartilages, peripheral extremities, and retinas. In addition, according to the method of the present invention, patterns for ejection of scaffolds and cells can be freely controlled. Thus, a composite cell tissue composed of a plurality of cell tissues such as skin tissue comprising blood vessels can be produced. A cell tissue produced by the method of the present invention can maintain its three-dimensional structure because of the presence of scaffolds and contact between cells. That is to say, the cell tissue of the present invention is a self-supporting tissue.

The obtained cell tissue can be used for transplantation, the repair of damaged tissue, and the like. For instance, it becomes possible to produce a skin sample comprising blood vessels by the method of the present invention, following which such skin sample can be transplanted to a damaged skin portion.

The size of the tissue produced by the method of the present invention is not limited, and it can be appropriately determined depending on the type of tissue to be produced. However, the surface area thereof is generally at least 0.1 cm², preferably at least 0.5 cm², and more preferably at least 1 cm² or at least 2 cm². Also, the thickness of tissue to be produced (height of a tissue produced on a substrate in a three-dimensional form) is not limited, and thus it can be appropriately determined depending on the type of tissue to be produced. However, it is generally at least 10 μm, preferably 20 μm, and more preferably 100 μm, 200 μm, or 500 μm.

The present invention also encompasses an artificial cell tissue having a three-dimensional structure, which is produced using the above pattern generator, and in which cells remain alive and neighboring cells are in contact with each other and the three-dimensional structure is maintained by scaffolds.

The present invention is hereafter described in greater detail with reference to the following example, although the present invention is not limited thereto.

The UE6E7T-12 (JCRB1151) mesenchymal stem cell line was obtained from the JCRB Cell Bank of the National Institute of Biomedical Innovation and cultured in a DMEM medium containing 10% fetal bovine serum. When the cells became approximately 80% confluent, they were detached with the use of trypsin, separated from each other, and suspended in a culture solution. Thus, a solution was prepared. Next, as shown in FIG. 1, a syringe into which the solution containing cells had been introduced and a syringe into which a solution containing scaffolds serving as cell footholds (a solution obtained by diluting 3 g of gelatin (from bovine bone, 074-02761, Wako) with 10 cc of a culture solution) had been introduced were positioned with a gap therebetween in a manner such that they were pointed at a dish containing a culture solution. Patterning was carried out with each solution by applying a high voltage to the relevant syringe via the flow of a corona discharge current as shown in FIG. 2.

A voltage waveform generated by a PC and a function generator (Iwatsu, Tokyo, SG-4105) was amplified with a high voltage amplifier (Matsusada Precision Inc., High voltage supply, HEOPS-5B6) in each syringe for patterning of scaffolds and cells (FIGS. 4A and 4B). In addition, transfer signals were transmitted by a driver (CHUO SEIKI, QT-K) or a PC for controlling the position of a dish via an XY linear stage controller (CHUO SEIKI, 2-axis stage controller, QT-CM2-35) and a stage (CHUO SEIKI, ALS-301-HM). Specifically, the scaffolds were first subjected to patterning in a desirable shape. Further, patterning of cells was carried out on the side of the scaffold pattern or thereon. After drawing of a single layer, the height of a culture solution filling a dish was increased by the height of a single layer. This operation was carried out repeatedly such that cell tissues were accumulated three-dimensionally. Then, patterning was carried out at an applied voltage of 0.5 kV to 5 kV. A Taylor cone was formed in a triangular pyramid shape on the syringe tip when a high voltage was applied. The tip portion of the Taylor cone was exclusively removed and ejected in a droplet form. At such time, the gap was set at 0.5 mm so as not to cause spark discharge. In such a case, if the gap exceeds 10 mm, floating droplets would be swept away by the surrounding air flow, resulting in a reduction of patterning accuracy.

Although a high voltage was applied, survival of the cells was confirmed after deposition, as shown in FIGS. 4C and 4D. Also, contact between ejected cells was confirmed.

FIG. 5 shows results obtained by drawing with a liquid containing scaffolds about 10 times at an applied voltage of 2.5 kV with a gap of 3.0 mm, followed by another instance of drawing with a liquid containing cells thereon at the same applied voltage with the same gap by the above technique. As shown in FIG. 5, cell tissue can be three-dimensionally formed.

In addition, in this Example, cells are ejected using a capillary tube with an inner diameter of 80 μm. However, in consideration of cell size, a capillary tube with an inner diameter of 30 μm can be used.

### INDUSTRIAL APPLICABILITY

The pattern generator for cell tissue patterning of the present invention is an apparatus using an electrostatic ink jet phenomenon, whereby even a highly viscous solution containing a scaffold and a solution containing cells can be ejected onto a substrate with high accuracy. The use of this apparatus allows patterning of cells that constitute tissue and a scaffold that serves as a foothold for the cells in an arbitrary pattern. Accordingly, a cell tissue having an arbitrary three-dimensional structure can be produced. In addition, the apparatus of the present invention makes it possible to eject cells maintained in a viable state and to establish adhesion between neighboring cells of the produced cell tissue. Therefore, artificial cell tissue that is similar to a real biological tissue can be produced. The thus produced tissue can be used for research, transplantation, and the like.

All publications, patents, and patent applications cited herein are incorporated herein by reference in their entirety.

What is claimed is:

1. A method for producing a cell tissue having a three-dimensional structure in which cells remain alive by patterning a cell tissue on a substrate using the pattern generator for cell tissue patterning on the substrate using an electrostatic ink jet system, which comprises
at least one vessel for holding a solution containing materials used for cell tissue formation,
a substrate that is an object of patterning,
a voltage applying unit for applying a voltage to the vessel, and
a plate electrode that is positioned between the vessel and the substrate, grounded, and perforated, the method comprising:
ejecting scaffolds and cells onto the substrate so as to allow the scaffolds and the cells to adhere to the substrate for patterning while allowing the cells to come into contact with the scaffolds; and
applying a voltage of 0.5 kv to 5 kv between the vessel and the plate electrode by the voltage applying unit in order to generate an electric field between the vessel and the plate electrode, causing the solution to be ejected from the vessel as a result of an electrostatic ink jet phenomenon and allowing the ejected solution to pass through a hole formed in the plate electrode and adhere to the substrate,
wherein a cell tissue is produced, the cell tissue having a three-dimensional structure, in which neighboring cells adhere to each other and cells remain alive, and
wherein the three-dimensional structure is maintained by the scaffolds.

2. The method according to claim 1, wherein the cell growth factor is further ejected in a manner so as to allow the cell growth factor to come into contact with cells.

3. The method according to claim 1, wherein the three-dimensional structure is formed by repeating patterning of scaffolds and cells so that a plurality of layers of scaffolds and cells are accumulated.

4. The method according to claim 1, wherein two or more types of cells are used.

5. The method according to claim 1, wherein the temperature of the vessel for holding a scaffold is maintained at 37° C. or higher and the temperature of the surface of the substrate is maintained at 37° C. or lower.
